(19) United States
(12) Patent Application Publication
(10) Pub. No.: US 2020/0253781 A1
(43) Pub. Date: Aug. 13, 2020

(54) LIQUID PATIENT INTERFACE

(71) Applicant: Carl Zeiss Meditec AG, Jena (DE)

(72) Inventors: Christian PÖSSEL, Weimar (DE); Thomas WOLLWEBER, Eckartsberga (DE); Tobias DAMM, München (DE); Michael Stefan RILL, Jena (DE); Dirk PREUSS, Jena (DE)

(73) Assignee: Carl Zeiss Meditec AG, Jena (DE)

(21) Appl. No.: 16/642,664

(22) PCT Filed: Sep. 4, 2018
(86) PCT No.: PCT/EP2018/073743

§ 371 Date: Feb. 27, 2020

(30) Foreign Application Priority Data
Sep. 5, 2017 (DE) 10 2017 215 589.2

(51) Int. Cl. A61F 9/009

(52) U.S. Cl. CPC A61F 9/009; A61F 2009/00851

(57) ABSTRACT

A liquid-patient interface for fixing the relative geometric position and orientation of a patient's eye with respect to a laser applicator of an ophthalmological laser therapy system. The liquid-patient interface includes a lens element and a cone element, wherein the lens element is inserted into the cone element and permanently connected to the cone element such that the liquid-patient interface has an integral configuration. The invention furthermore relates to a corresponding production method for such a liquid-patient interface. The liquid-patient interface, the lens element of which is embodied in one piece and contains an optical zone, which has a lens function, and an envelope region, adjoining the optical zone, having a defined height not equal to zero and having an upper edge, wherein the upper edge of the lens element facilitates a direct connection to the laser applicator.

### RELATED APPLICATIONS

This application is a National Phase entry of PCT Application No. PCT/EP2018/073743 filed Sep. 4, 2018, which application claims the benefit of priority to DE Application No. 10 2017 215 589.2, filed Sep. 5, 2017, the entire disclosures of which are incorporated herein by reference.

### TECHNICAL FIELD

The present invention relates to a liquid-patient interface for fixing the relative geometric position and orientation of a patient's eye with respect to a laser applicator of an ophthalmological laser therapy system.

### BACKGROUND

In laser-surgical applications, especially in laser-assisted cataract surgery, the relative geometric position and orientation of the patient's eye with respect to the laser focus and hence with respect to the employed ophthalmological laser therapy system must be accurately defined. However, since eye-drop anesthesia is used, as a rule, in an operation, the patient can, however, move their eye voluntarily or involuntarily—the latter movements are referred to as microsaccades. In order to fix the relative position and orientation of the eye and laser therapy system, the patient's eye is therefore mechanically coupled to the laser therapy system with the aid of a patient interface (PI).

In laser-assisted cataract surgery, in particular, so-called liquid-patient interfaces have proven their worth. Historically, such devices have consisted of a funnel-shaped container which, as a rule, is filled with physiological saline solution (BSS, buffered salt solution). Moreover, they contain an optical element, a patient interface lens, which forms the distal end of the laser optical unit of the ophthalmological laser therapy system. This laser optical unit focuses and guides the laser beam. In this case, the distal end of the laser optical unit is immersed in the saline solution. This arrangement has advantages over rigid patient interfaces such as, e.g., contact glass, for example, there is only a slight increase in intraocular pressure when liquid-patient interfaces are used. Moreover, the formation of corneal folds, which could impede the optical imaging of the laser beam in the lens of the patient's eye, is avoided. Additionally, the use of a liquid-patient interface allows the refractive index between the laser optical unit and the corneal material of the patient's eye to be adapted.

The entire liquid-patient interface, or at least the parts of the liquid-patient interface that are in direct contact with the patient's eye, should be sterile and because the eye can react very sensitively to bacterial or viral infections. An infection can quickly lead to a loss of sight.

Thus, in laser-assisted eye surgery, patient interfaces may fulfil two or more tasks, for example, they can mechanically fix the patient's eye to a laser applicator of an ophthalmological laser therapy system, by application of which the laser radiation is emitted into the eye. Moreover, they are the last optical element of the laser optical unit, in particular of a laser objective lens. Therefore, as decisive interface element, they must satisfy both high mechanical and high optical requirements.

Currently available liquid-patient interfaces consist of a plurality of parts, which have to be assembled during the surgical operation. This means that they have to be assembled by the assistant or the surgeon prior to use and/or are brought together during coupling to the patient's eye. Sterility must be maintained at all times in the process. Since assembly is not simple, this may cause the liquid-patient interface to be contaminated prior to use. Therefore, the aforementioned requirements can only be met with difficulties by such a liquid-patient interfaces. Examples of such multi-part liquid-patient interfaces are described in documents US2010/0274228 A1, US 2011/0022035 A1 and US2016/0175146 A1.

Moreover, bringing the patient-interface lens to the same position every time in reproducible fashion is difficult in the case of multi-part liquid-patient interfaces. Since the patient-interface lens is an important part of the laser optical unit and incorrect or inaccurate positioning can have a direct effect on the optical quality of the overall system, this reproducible positionability is essential to the success of the therapy. Many mechanical connection techniques, such as, e.g., grooves and tongues, click connections, clamping connections, etc., have great distance tolerances between the connected elements in contrast to adhesive bonding, welding and casting connections. Therefore, conventionally, pre-assembled liquid-patient interfaces are more precise than liquid-patient interfaces that are only assemblable during the operation.

WO 2016/058931 A2 describes an integral liquid-patient interface that is couplable to the ophthalmological laser therapy system by the surgeon in very simple fashion, using one hand and without requiring assistance. In principle, such handling of a liquid-patient interface that is simple is advantageous. However, care has to be taken that the manufacture of such integral liquid-patient interface, in which the assembly of possible individual parts such as the patient interface lens with the funnel-shaped container has already been carried out, is implemented very precisely. In turn, this increases the complexity for the manufacture of the liquid-patient interface.

### SUMMARY OF THE INVENTION

The disclosure describes a liquid-patient interface and a method for producing such a liquid-patient interface, which is conceptually simple, user-friendly and efficiently employable within daily use within clinical practice but, can also be easily produced such that the high demands on the technical precision are reliably met.

In some embodiments, the disclosure describes a liquid-patient interface for fixing the relative geometric position and orientation of a patient's eye with respect to a laser applicator of an ophthalmological laser therapy system, said liquid-patient interface comprising a lens element and a cone element, wherein the lens element is inserted into the cone element and permanently connected to said cone element in such a way that the liquid-patient interface has an integral configuration. The disclosure furthermore relates to a corresponding production method for such a liquid-patient interface.

### BRIEF DESCRIPTION OF THE DRAWINGS

The present invention will now be explained on the basis of the following embodiments.

In the drawing:

FIGS. 1a to 1c show a lens element 2 of an embodiment of the liquid-patient interface according to the invention in different views;

FIGS. 2a to 2c show a cross section through an applicator interface of a laser applicator of an ophthalmological laser therapy system and through an embodiment of a liquid-patient interface according to the invention,

FIG. 3 shows a view of another embodiment of the liquid-patient interface according to the invention, and

FIG. 4 shows an ophthalmological laser therapy system with a fixed liquid-patient interface according to the invention.

### DETAILED DESCRIPTION

In some embodiments, the disclosure describes a liquid-patient interface for fixing the relative geometric position and orientation of a patient's eye with respect to a laser applicator of an ophthalmological laser therapy system comprises a lens element and a cone element. As described above, a liquid, such as BSS, is filled between the lens element and a patient's eye in such a liquid-patient interface after said liquid-patient interface has been placed on and fixed to the patient's eye.

The lens element may be inserted into the cone element and permanently connected to said cone element in such a way that the liquid-patient interface has an integral configuration.

In some embodiments, inserting the cone element into the lens element can be made easier if a shoulder on which the lens element can be placed is provided in the cone element—thus, such a shoulder in the cone element is advantageous. However, a precise deposition may also be possible by way of an alternative design of the lens element with respect to the cone element. The fit between the cone element and the lens element should not have too tight of tolerances since mechanical forces on the lens element may lead to undefined changes in the optical properties.

By contrast, the relative position and orientation of the cone element with respect to the lens element is less critical on account of the design of the disclosed liquid-patient interface.

In some embodiments, an integral configuration of the liquid-patient interface means that all parts of the liquid-patient interface are permanently connected to one another such that an assembly in the operating theater (OP) prior to use of the liquid-patient interface is dispensed with and the liquid-patient interface can be used without any further assembly to fix the relative position and orientation of the patient's eye with respect to a laser applicator of an ophthalmological laser therapy system. In this way, the use of this liquid-patient interface is possible even without help by an assistant and the configuration outlay prior to an operation is very low.

In some embodiments, the disclosed liquid-patient interface may be characterized in that the lens element is embodied in one piece. “In one piece” (in contrast to “integral”) means that the lens element is manufactured from one piece.

The disclosed lens element comprises an optical zone, i.e., a region through which the laser beam propagates during use. The optical zone is transparent and contains an optical function that has a lens function. Furthermore, the lens element comprises an envelope region adjoining the optical zone, said envelope region advantageously likewise having a slightly conical embodiment with a defined height not equal to zero and an upper edge, wherein the upper edge of the lens element facilitates a direct connection to the laser applicator.

The height of the envelope region of the lens element defines the effective distance between the last lens of the laser applicator of the ophthalmological laser therapy system and the upper boundary or interface of the optical zone of the lens element: The upper edge of the lens element facilitating a direct connection to the laser applicator of the ophthalmological laser therapy system means that this upper edge is brought into direct contact with the laser applicator without further intermediate elements and, as a consequence, a direct connection is established between the lens element of the liquid-patient interface and the laser applicator of the ophthalmological laser therapy system. In some such examples, the image field is not restricted by the connection to the laser applicator by way of the envelope region having a slightly conical embodiment and the adjoining upper edge.

Hence, the lens element is configured to precisely and repeatably define the relative geometric position and orientation of the last optical applicator element of the laser applicator of the ophthalmological laser therapy system with respect to the optical zone of the lens element.

This, in turn, sometimes renders it possible to precisely and repeatably determine the relative geometric position and orientation of a laser focus generated in an ophthalmological laser therapy system with respect to a patient's eye. Here, “precise” means with significantly smaller deviations in comparison with the prior art since no intermediate elements are used in comparison with the prior art, which otherwise would require some manufacturing tolerances. The position and orientation of the laser focus with respect to the patient's eye is “repeatable” because the positioning of the optical zone of the lens element of the liquid-patient interface with respect to the last optical element of the laser applicator can be implemented in the same way, once again on account of the direct fixation of the lens element to the laser applicator. Hence, the overall optical system determining the laser focus in terms of its structure, position, and orientation is defined in precise and repeatable fashion. By contrast, the distance of the optical zone of the lens element of the liquid-patient interface from the patient's eye is uncritical as the latter is set individually during operation planning, for example by way of OCT (optical coherence tomography) imaging.

Consequently, the mechanical interface for the direct contact and the direct connection of lens element to laser applicator and the height (h) of the envelope region of the lens element may be useful in this liquid-patient interface design. However, this direct connection of the lens element to the laser applicator is advantageous from a structural point of view since all critical requirements in respect of the manufacturing accuracy, e.g., in respect of the optical and mechanical parameters, are addressed in this one partial component part—the lens element—only. By contrast, were the liquid-patient interface to be coupled to the laser applicator via the cone element, the lens element would, in turn, have to be connected in mechanically defined fashion and with very high precision to the cone element. Then, there would be an additional interface with very tight tolerance limits. However, this is avoided by the design according to the invention: All critical parameters lie in the lens element itself.

Since the accuracy requirements in respect of the lateral and axial position of the optical zone of the liquid-patient interface relative to the laser optical unit of the ophthalmological laser therapy system are provided by the lens element, the manufacturing tolerances for the cone element becomes uncritical. As discussed above, the distance of the lens element from the patient's eye also becomes uncritical. As a result, the relative position and orientation of the cone element with respect to the lens element is uncritical as all critical requirements are unified in the integral lens element.

The structure of the liquid-patient interface according to the invention presented here therefore also allows a very uncomplicated therapy work flow:

During the therapy work flow, the liquid-patient interface is initially attached to the laser applicator of the ophthalmological laser therapy system. Then, the liquid-patient interface coupled to the laser applicator is placed directly onto the patient's eye and sucked on by way of vacuum suction via a suction lip, which is disposed at the lower edge of the patient interface cone. The interstice between the patient's eye and the lens element is then filled with a biocompatible liquid in order to level off the refractive index difference and hence reduce back-reflections and also increase the transmission and, secondly, to prevent the patient's eye from drying out during the procedure.

An example embodiment of the disclosed liquid-patient interface is characterized in that the upper edge of the lens element comprises a structure for forming a mechanically stable, direct connection to an applicator interface of the laser applicator of the ophthalmological laser therapy system.

The mechanical interface, e.g., the boundary surface, for the direct contact between the lens element and the laser applicator, in particular an applicator interface of the laser applicator, and for the direct connection therebetween is formed by the upper edge of the lens element. The latter has a corresponding structure for establishing a direct connection to the applicator interface, the manifestation and manufacturing precision of which is critical for this liquid-patient interface design. However, in turn, critical requirements in respect of the cone element, which are conventionally needed in the prior art, are dispensed with.

In various advantageous embodiments, the lens element of the liquid-patient interface according to the invention, in particular the structure of the upper edge thereof, is embodied in different ways in order to realize a mechanically stable, direct connection to an applicator interface of the laser applicator of the ophthalmological laser therapy system.

In some embodiments, the structure of the upper edge of the lens element comprises an end face for forming a vacuum-tight connection by way of vacuum suction onto the applicator interface, wherein the vacuum suction is implemented by evacuating a volume that is delimited by a last optical applicator element, the applicator interface and the lens element, the applicator interface having a vacuum suction channel into the volume to this end.

In other embodiments, the structure of the upper edge of the lens element comprises an end face for forming a vacuum-tight connection by way of vacuum suction onto the applicator interface, wherein the vacuum suction is implemented by way of the end face, the applicator interface having a vacuum suction channel that is positioned at the end face to this end. In this case, the vacuum suction channel should for example be able to suck over a large area region of the end face.

For both of these embodiments, the end face adjoins the applicator interface in flush fashion. By way of example, this is realized if the end face is configured to be plane parallel with respect to the applicator interface and is polished to be smooth.

In another embodiment, the upper edge of the lens element comprises a structure that facilitates an interlocking and/or force-fit connection to the applicator interface. Interlocking connections can be produced by application of at least two connection partners engaging in one another. As a result, the connection partners cannot come apart even when there is no force transmission or the force transmission is interrupted. Expressed differently, one connection partner is in the way of the other connection partner in the case of an interlocking connection. Force-fit connections assume a normal force on the faces to be connected to one another. Their mutual displacement is prevented as long as the counterforce brought about by the static friction is not exceeded.

However, it is necessary to always ensure that the optical properties of the lens element, in particular of the optical zone of the lens element, do not change as a result of this connection to the applicator interface of the laser applicator. To this end, all tension should be avoided when establishing this connection.

In the case of the liquid-patient interface in which, according to the invention, a connection is obtained by way of the structure of the upper edge of the lens element, a connection that closes immediately and causes no wear from a mechanical point of view is moreover advantageous. Examples of these include a “click connection” to the applicator interface, e.g., the liquid-patient interface, in particular the lens element of the liquid interface, is placed on the applicator interface and brought into a fixed position by way of a short mechanical movement, in which the structure of the applicator interface and a structure of the upper edge of the lens element latch into one another. This connection thus is based on a lock-and-key principle and the structure of one side—e.g., the applicator interface—hence defines the structure of the other side—e.g., the upper edge of the lens element—, or vice versa. Clamping connections are also conceivable, provided that only a pressure that is negligible for the optical zone of the lens element is exerted.

In some embodiments, the disclosed liquid-patient interface is moreover characterized in that the upper edge of the lens element comprises a positive alignment structure, such as a shoulder, which is configured to engage in a negative alignment structure disposed at the applicator interface.

It may be advantageous if the lens element of the liquid-patient interface facilitates an additional lateral alignment in order to be able to highly precisely align the liquid-patient interface, in particular the lens element, with respect to the applicator interface and, ultimately, with respect to the aperture of the laser applicator, even in the lateral direction. This can be ensured by the described alignment structure.

A corresponding negative alignment structure on the applicator interface in relation to the shoulder in the upper edge of the lens element is realized, for example, by one or more pins or a negative shoulder fitting to the shoulder of the upper edge of the lens element.

It may be advantageous if the lens element of the liquid-patient interface according to the invention consists of a polymer, such as polycarbonate, or an optical glass, such as silicon dioxide. The optical material of the lens element must offer high transparency to therapy wavelengths and examination wavelengths (e.g., OCT wavelengths). If used in refractive surgery or, for example, in cataract surgery, pulsed laser radiation, typically from a femtosecond laser source or a picosecond laser source, is usually used as therapy laser radiation. Typical examination wavelengths, in turn, are all wavelengths used an optical coherence tomography, but also the wavelengths of visible light and infrared radiation.

Lens elements made of polymer are advantageous in that they can be manufactured in very high numbers and reproducible fashion by for example injection molding methods. In this case, the production costs are substantially lower than in the case of glass lenses. By contrast, glass lenses can be produced more precisely using conventional methods while polymer lenses lead to higher tolerance deviations.

Polymer lenses can be produced in large numbers and cheaply, e.g., by an injection molding method. Here, the geometric form to be molded is converted into a mold as a negative. As a rule, this mold consists of chemically stable and dimensionally stable materials, such as, e.g., steels or engineering ceramics, that allow many molding cycles and, in the process, always meet the requirements in respect to the given tolerances. In the case of an injection molding method, the polymer granulate is initially thermally liquefied and pressed through a hot runner into the injection mold at a high pressure (approximately 500 to 2000 bar). The location at which the polymer melt penetrates into the mold is referred to as a gate. Proceeding from the gate, the liquid polymer is distributed in the mold. A nonreturn valve prevents the polymer melt from flowing back in the direction of the gate. Attempts are made to obtain a flow behavior of the melt that is as laminar as possible during the injection; i.e., the melt is immediately cooled and solidifies at the mold edge. The following melt is pressed at even higher speeds through the melt channel, which has been tapered as a result thereof, and subject to a stretch deformation toward the edge, forward at the melt front. In some embodiments, cooling of the polymer melt leads to loss of volume, which has an effect on the dimensional accuracy and surface quality of the molded part.

For this reason, manufacturing lens elements from polymers using an injection molding method incidentally may be advantageous for the production of conventional so-called “contact glasses”, i.e., patient interfaces or contact apparatuses in which the optically active zone of the lens element is placed directly on the cornea of the patient's eye and there consequently is no need for filling with a saline solution. Usually, such contact glasses are used in refractive laser surgery for the treatment of the cornea—e.g., where the laser beam, for example a femtosecond laser beam, need not penetrate so deep into the eye structures but work is carried out near the surface. In this case, too, a defined distance of the optical zone of the lens element from the first optical element of a laser applicator of an ophthalmological laser therapy system is very important. Here, the precision of the configuration of the lens element itself is of even greater importance: The surface accuracy must be very high and the refractive index distribution should be absolutely stable over the entire lens element.

On account of the substantially lower penetration depth of the focused laser beam, lens elements of a conventional contact glass, may require no (conical) envelope region, adjoining the optical zone, with a defined height not equal to zero or no separate upper edge in order to facilitate a direct connection to the laser applicator and in order to nevertheless obtain a diameter of the image field that is so wide that problem-free working is possible, in particular in the entire cornea of the patient's eye.

Thus, in some embodiments by using an injection molding method, a contact glass and a method for producing such a contact glass, which is conceptually simple, user-friendly and efficiently employable within daily use within clinical routine but, at the same time, also easily producible so that the high demands on the technical precision are reliably met, is described as follows:

A contact glass for fixing the relative geometric position and orientation of a patient's eye with respect to a laser applicator of an ophthalmological laser therapy system, comprising a lens element and a cone element, also referred to as contact glass holder, wherein the lens element is inserted into the cone element and permanently connected to said cone element in such a way that the contact glass has an integral configuration and wherein the lens element
is embodied in one piece and has an optical zone with a lens function which may extends over the diameter of the lens element,
may be adapted to the curvature of the cornea on the side of the eye and is configured to be plane on the side distant from the eye,
has a circumferential edge region with a constant thickness not equal to zero, characterized in that the lens element is formed from a polymer by way of an injection molding method.

In some such embodiments, the cone element only comprises a wall, a lower suction lip and a vacuum feedthrough because the lens element is situated directly on the cornea of the patient's eye when the contact glass is applied for fixing the position of the eye with respect to the laser applicator of the ophthalmological laser therapy system. Filling with physiological saline is not necessary in this case.

Hence, the disclosure also includes a patient interface or a contact apparatus for fixing the relative geometric position and orientation of a patient's eye with respect to a laser applicator of an ophthalmological laser therapy system, which is embodied as a contact glass or as a liquid-patient interface comprising a lens element and a cone element, wherein the lens element is inserted into the cone element and permanently connected to said cone element in such a way that the contact glass has an integral configuration and wherein the lens element
is embodied in one piece,
has an optical zone with a lens function,
has a defined height not equal to zero, said height precisely determining the distance of the last optical element of the laser applicator of the ophthalmological laser therapy system from the emergence location of the laser beam from the optical zone of the lens element,
characterized in that the lens element moreover comprises an edge region, which facilitates a direct connection to the laser applicator and which is formed from a polymer by an injection molding method.

Such a solution succeeds in producing a patient interface—both a contact glass and a liquid-patient interface—with high precision since the lens element in both variants is the only element of the patient interface that must be produced with highest dimensional accuracy and optical quality as it unifies all critical requirements in respect of the patient interface in itself. By contrast, the cone element, in which the lens element is then received, and the process of assembling the two elements may have substantially high manufacturing tolerances. By producing the lens element using an injection molding method, a simple and high-volume production of this lens element is possible.

If the lens element of the liquid-patient interface or of a contact glass is formed from a polymer by an injection molding method, it may be advantageous if the gate mark is disposed outside of the optical zone, as there would otherwise be restrictions in the usable region of the optical zone.

In some embodiments, it may be advantageous if the gate mark of a lens element of the liquid-patient interface, formed by an injection molding method, is disposed at a side of the upper edge of the lens element where the latter has a maximum diameter, with this side being beveled in such a way that a pin arising during molding does not exceed the maximum diameter of the upper edge. Were the gate mark to exceed the maximum diameter, the lens element would jam during assembly in the cone element, possibly altering the optical properties of the optical zone of the lens element in undefined fashion.

However care should be taken that the gate mark does not impede the direct connection of the lens element to applicator interface of the laser applicator. By way of example it should not disturb the structure of the upper edge, especially not if vacuum suction is provided via an end face of the upper edge.

In another example embodiment, the gate mark of a lens element formed by an injection molding method is disposed at the inner side of the upper edge or at the outer side of the envelope region, in particular the outer side of the lower portion of the envelope region.

In principle, the wall strengths of the lens element can be chosen as desired. In the case of injection molded lens elements, structures with virtually constant wall strength are particularly suitable since the liquid polymer melt is distributed more uniformly in this case and shrinkage of the material during cooling can be better compensated in advance. The optical properties become calculable as a result of uniform shrinkage.

In some embodiments, the lower delimiting face of the optical zone of the lens element of the liquid-patient interface, e.g., the face facing the patient's eye, is embodied as an optical element of the liquid-patient interface and has, in particular, a lens function. As a consequence, as a last optical element, it is also responsible for the definition of the laser focus in terms of the structure and position and orientation thereof. When the lens element is connected to the applicator interface of the laser applicator of an ophthalmological laser therapy system, the optical zone of the lens element is consequently part of an epi overall optical system for shaping and positioning the laser focus. As result of the option of precise and repeatable positioning, it consequently also contributes to the qualitatively high quality shaping and positioning of the laser focus.

A lens element of the liquid-patient interface whose upper delimiting face of the optical zone, e.g., the side facing away from the patient's eye, has an antireflection layer or an antireflection layer system may be advantageous.

An antireflection layer reduces the reflections at the interface of the lens element to the air or to the vacuum, which reflections would arise because there usually is a great difference here in the refractive indices between air or vacuum and the material of the lens element. Consequently, it avoids back reflections—of the high-energy laser radiation, for example—into the laser applicator, which could destroy internal components such as, e.g., lenses, prisms, detectors, etc. Here, work is very frequently carried out not only with an isolated antireflection layer; instead, use is made of an antireflection layer system. In the present case, such an antireflection layer system has a freely choosable layer sequence or a layer sequence adapted to the requirements.

If an antireflection layer or an antireflection layer system is used on the upper delimiting face of the lens element of the liquid-patient interface according to the invention, then it is embodied such that the reflection of radiation in at least one of the following wavelength ranges is suppressed, wherein a suppression in the following wavelength range is implemented with a reflection (R) specified for the wavelength range:
for therapy laser radiation, wavelengths from 1000 nm to 1100 nm with reflection R of less than 1%;
for OCT laser radiation, wavelengths from 800 nm to 1200 nm with reflection R of less than 1%;
for the use of infrared light (when infrared cameras are used), wavelengths from 800 nm to 1000 nm with a reflection R of less than 10%;
for the use of light in the visible range, in particular if the liquid-patient interface is used in conjunction with a laser therapy system that uses a surgical microscope whose surgical microscope head is couplable to a laser applicator such that, already as a result thereof, visible light strikes the eye of the patient through the liquid-patient interface, wavelengths from 400 nm to 700 nm with a reflection R that remains as constant as possible over this wavelength range, e.g., without significant minima or maxima, such that no significant changes of the absorption or reflection occur in the visible range, and no color distortions arise when looking through the optical zone of the lens element.

Since a combination of the aforementioned radiations is often used for examination and for therapy, the antireflection layer or the antireflection layer system must be configured in such a way that the light from the various employed and aforementioned spectral ranges is reflected as little as possible. As already mentioned, the specific layer sequence of this antireflection coating, e.g., both the material and the thickness, is freely choosable such that a reflection in the critical wavelength range or in the critical wavelength ranges (see above) is suppressed or, as specified above, minimized. However, the selection of the optimal layer materials also depends strongly on the material of the lens element itself since aspects such as adhesion, biocompatibility, matching to the laser wavelength, etc., must be taken into account in the given medical surroundings.

In some embodiments, it may be advantageous if the lens element of the liquid-patient interface is configured to receive, at an end side of the upper edge, which may be polished to this end, an illumination output coupling from the applicator interface and/or if the cone element—in an alternative variant or a variant usable in parallel—is configured to locally form a direct contact with the applicator interface, when this mechanical coupling point should likewise be polished in order thereby to receive illumination output coupling from the laser applicator.

In an embodiment of an ophthalmological laser therapy system, the laser applicator is used together with a surgical microscope (OPMI). In one variant, the observation path in this case leads from the surgical microscope through the optical unit of the laser applicator to the patient's eye. As a rule, the illumination path of the surgical microscope is guided coaxially with, or near to, the observation path. In this case, the illumination path also crosses the optical zone of the lens element, which may in turn lead to bothersome reflections in the image. In alternative variants, which may possibly also be usable in parallel, the illumination of the patient's eye can now be designed in such a way in this case that said illumination is coupled laterally into the lens element at a location that does not belong to the optical zone or else that the illumination of the eye is implemented by way of the cone element.

A further embodiment of the liquid-patient interface according to the disclosure is characterized by a cone element which comprises a cone wall, a lower suction lip, a vacuum feedthrough, and a filling channel for liquids, wherein the vacuum feedthrough extends through the cone wall into the suction lip and the filling channel for liquids extends through the cone wall into a second volume formed by the patient's eye, the cone wall and the lens element in the case of vacuum suction.

For the purposes of fixing the liquid patient interface, the vacuum feedthrough is then connected to a vacuum pump by a tube while the filling channel is connected to a corresponding liquid reservoir.

In some embodiments, it may be advantageous if the lens element is adhesively bonded to the cone element of the liquid-patient interface with the aid of an adhesive that exerts no tensile or warping forces during the drying process. As result of such occurring forces, the lens element would be deformed and thus change its optical properties in undefined fashion such that a figure (e.g., a figure defect) and/or astigmatism could arise. The adhesive and all component parts directly or indirectly touching the patient must moreover be biocompatible.

Moreover, it is particularly expedient if the cone element of the liquid-patient interface comprises a collar that is embodied as an extension of the cone wall. As a result, the sterile region of the laser applicator, particularly in the critical part near the patient's eye, is enlarged following an attachment of the liquid-patient interface to the laser applicator of an ophthalmological laser therapy system, for example, the collar goes beyond the applicator interface and envelops the latter or the entire lower part of the laser applicator up to a height set by a collar height.

In some embodiments, the collar of the cone element does not nestle directly against the laser applicator when fastening the liquid-patient interface to the laser applicator but instead leaves a small gap such that a drape can optionally be introduced between the laser applicator and liquid-patient interface.

For reliably filling the liquid-patient interface, it may be advantageous if the filling channel is disposed in the cone element with such an offset in relation to the vacuum feedthrough that filling channel and vacuum feedthrough do not coincide in a view on the liquid-patient interface from above.

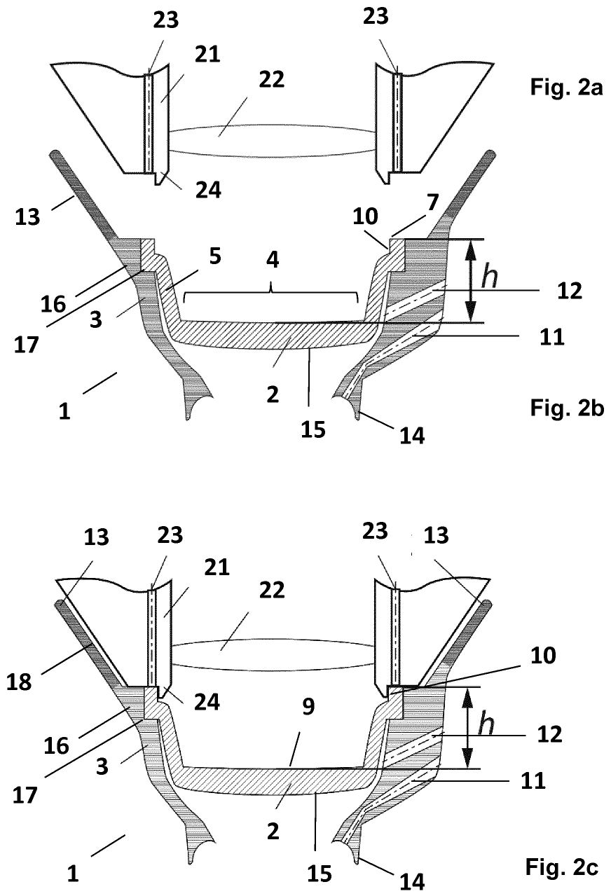

As depicted in FIGS. 2b and 2c, the distance between the cone element 3 and the lens element 2 can be kept very small for structural reasons at the usual position of the filling channel 12 for liquids. According to some examples in the prior art, the filling channel 12 and the vacuum feedthrough 11 were previously disposed one above the other since this allows the supply tubes to these channels 11, 12 to be guided together, which generally are likewise pre-assembled at the liquid-patient interface 1, and so these only still have to be connected to a vacuum pump and a liquid reservoir, respectively. If these are guided through the cone wall at completely different sides, this would, in turn, make fixing the liquid-patient interface more difficult on account of the individual hanging tubes. However, on the one hand, the external diameter of the cone element 3 should be as small as possible such that the liquid-patient interface can be used in as many patients as possible, i.e., also in the case of small and low-lying eyes. On the other hand, the optical zone 4 of the lens element 2 should be as large as possible in order to have an optically effective region that is as large as possible available for the therapy. A problem that may arise as a result thereof is that, in the case of liquids with a high viscosity, the liquid does not flow into the volume between the lens element 2 and the patient's eye but is drained laterally as a result of surface tension.

In some embodiments, attempts have to be made to design the distance between the cone element 3 and the lens element 2 to be as large as possible. Therefore, the filling channel 12 in the cone element must be pulled down as far as possible (e.g., in the direction of the patient's eye) such that the liquid-patient interface 1 is filled with the liquid through the filling channel 12 which is disposed below the lower boundary face of the optical zone 4 of the lens element 2 in the ideal case and the position and orientation of which in reality at least approximate this ideal case. However, if the liquid-patient interface 1 is coupled to the patient's eye 50 by application of the vacuum, a constructive collision arises here. On account of the application, the two channels 11 and 12 must not cross or overlap.

A mutually offset arrangement of filling channel for the liquid, e.g., BSS, and the vacuum feedthrough such that both channels are still disposed in the vicinity of one another but no longer coincide in a view from the top allows the supply tubes still to be guided together but allows the filling channel 12, for filling the liquid, to be moved through the cone wall into a region of the cone element 3—further down—where the distance between the cone element 3 and the lens element 2 is significantly larger than if the filling channel 12—as was conventional—is disposed above the vacuum feedthrough 11.

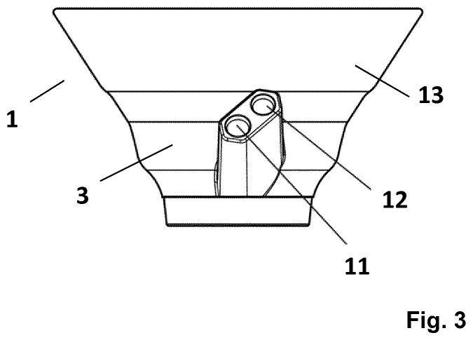

Thus, as shown in FIG. 3, the filling channel 12 is offset and disposed downward in relation to the vacuum feedthrough 11. This also allows viscous fluids to be poured.

The liquid-patient interface according to the invention is therefore simple, user-friendly and efficiently usable for daily use within clinical routine on account of its special features described here, and it facilitates handling during the operation by a single person (e.g., the surgeon or an assistant). Moreover, it is optimized for high technical precision in respect of its producibility and reliable use, said precision being obtained by controlling significantly fewer critical parameters (in terms of number) and simpler process steps in comparison with liquid-patient interface according to the prior art since all critical parameters are concentrated in the one-piece lens element of the liquid-patient interface and consequently different parts of the liquid-patient interface no longer need to be produced highly precisely and be aligned highly precisely with respect to one another.

In a production method according to the invention for a special liquid-patient interface for fixing the relative geometric position and orientation of the patient's eye with respect to a laser applicator of an ophthalmological laser therapy system, a lens element containing an optical zone, which has a lens function, and an envelope region, adjoining the optical zone, with an upper edge is initially manufactured in one piece, such as from a polymer in an injection molding method.

Thereupon, the manufactured lens element is inserted in and adhesively bonded with an integral cone element, which comprises a cone wall, a lower suction lip, a vacuum feedthrough, and a filling channel for liquids.

In some embodiments, the upper edge of the lens element is manufactured in such a way in this case that it facilitates a direct connection to the laser applicator. To facilitate this, a structure may be formed at the upper edge of the lens element, said structure facilitating a mechanically stable, direct connection to an applicator interface of the laser applicator.

Therefore, in the production method according to the invention for a liquid-patient interface, which is particularly simple if an injection molding method is used to produce the lens element, only a few critical parameters have to be taken into account in a method that, in principle, is short.

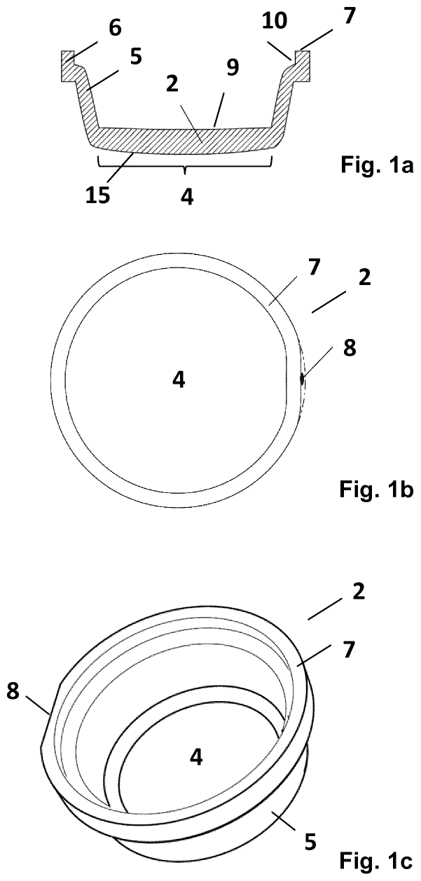

FIGS. 1a to 1c illustrate a lens element 2 of an example liquid-patient interface 1 according to the invention in different views: in a sectional view from the side (FIG. 1a), in a plan view of end face 7 of the upper edge 6 of the lens element 2 (FIG. 1b) and in a projection view (FIG. 1c). In some example embodiments, the lens element 2 is manufactured in one piece from a polymer, specifically from polycarbonate, in an injection molding method. In an injection molding method, a molded part is cast through a filling opening. In the process, a gate mark 8 arises, usually in the form of a pin. The lens element 2 comprises an optical zone 4, which is adjoined by an envelope region 5. This envelope region 5 has a defined height h, which determines the distance of the optical zone 4 of the lens element 2 from a last optical applicator element 22 of a laser applicator 220, not shown in this figure, of an ophthalmological laser therapy system. The upper edge 6 of the envelope region 5 has a flat end face 7 (which is polished to this end) for vacuum suction onto an applicator interface 21 of the laser applicator 220 and a shoulder 10 for the lateral alignment with respect to the applicator interface 21. The optical zone 4 has a lens function 15. In order to avoid reflections at the lens element 2 and hence mirroring of examination radiation of an OCT laser and, in particular, of high-energy therapy laser radiation into the laser applicator 220 of the ophthalmological laser system 100 during use of said ophthalmological laser therapy system 100, the lens element 2 has an antireflection layer 9 at its upper delimiting face.

As shown in FIG. 1, the gate mark 8 of the lens element 2 is disposed at the outer upper edge 6—as illustrated in the plan view on the lens element 2 in FIG. 1b. To this end, the outer upper edge 6 of the lens element 2 is bevelled in the region of the gate mark 8, to be precise in such a way that the pin-shaped gate mark 8 does not protrude beyond the maximum diameter of this upper edge 6, as indicated by the dash-dotted line in FIG. 1b. This bevel can also be seen in the projection view of FIG. 1c. If care is taken that the gate mark 8 does not protrude beyond the maximum diameter of the upper edge 6, no tensions arise as a result of the deformation of the lens element 2 when unifying lens element 2 and cone element 3 to form an integral liquid-patient interface 1. Otherwise, the lens element 2 would have to be pressed into the cone element 3 with the application of force, which could change the optical properties in undefined fashion as a result of the deformation arising in the process.

FIGS. 2a to 2c show a cross section through an applicator interface 21 of a laser applicator 220 of an ophthalmological laser therapy system 100 and through a disclosed liquid-patient interface. In this liquid-patient interface 1, which is illustrated in FIG. 2b in the uncoupled state vis-a-vis FIG. 2a showing an applicator interface 21 of a laser applicator 220 of an ophthalmological laser therapy system 100, the lens element 2 of FIGS. 1a to 1c has been inserted into a cone element 3 and permanently connected therewith. Finally, FIG. 2c shows a liquid-patient interface 1 in the coupled state at the applicator interface 21.

The cone element 3 comprises a wall 16, within which the lens element 2 has been inserted on, and by way of adhesive bonding permanently been connected to, a shoulder 17. A vacuum feedthrough 11 extends through the wall 16 into the suction lip 14 for vacuum suction of the liquid-patient interface 1 onto a patient's eye 50. Moreover, extending through the wall 16 there is a filling channel 12 for filling the volume between the lens element 2, the wall 16 of the cone element 3 and a patient's eye 50 with a refractive index-adapted liquid (e.g., a saline solution, BSS) following a vacuum suction of the liquid-patient interface 1 onto the patient's eye 50.

The wall 16 of the cone element 3 has been extended by a collar 13. In the state coupled to the applicator interface 21, this collar 13 is located around said applicator interface 21 in order to shield the letter from the patient's eye 50 and thus enlarge the sterile region. However, the collar 13 then does not rest directly on the applicator interface 21 but leaves a gap 18 into which a sterile cover (drape) can be introduced, the latter likewise serving for protection purposes and for enlarging the sterile region.

The applicator interface 21 of the laser applicator 220 of an ophthalmological laser therapy system 100 comprises a vacuum suction channel 23, which, when the liquid-patient interface 1 is coupled to the applicator interface 21, rests on the end face 7 of the upper edge 6 of the lens element 2 of the liquid-patient interface 2, said end face being sucked thereagainst thereby. For the correct lateral alignment of the liquid-patient interface 2 with respect to the applicator interface 21 before vacuum suction, the applicator interface 21 comprises a pin structure 24. The latter engages into a shoulder 10 in the upper edge 6 of the lens element 2 of the liquid-patient interface 1.

The applicator interface 21, which is part of a laser applicator 220 of an ophthalmological laser therapy system 100, comprises a laser aperture through which therapy laser radiation can enter into the liquid-patient interface 1 and, from there, into the eye 50 of a patient.

Furthermore, the applicator interface comprises a last optical applicator element 22, which forms the last lens of the applicator objective lens in this case and which is part of the optical system for guiding and focusing therapy laser radiation of the laser therapy system 100 and which is possibly also part of an optical system for guiding and focusing examination radiation.

FIG. 3 shows a side view of another embodiment of the liquid-patient interface 1 according to the invention, in which an arrangement of the filling channel 12 with respect to the vacuum feedthrough 11 is illustrated at the outer wall 16 of the cone element 3 of the liquid-patient interface 1. The advantages of such an arrangement have been already described above: As is evident from FIGS. 2b and 2c, the distance between the cone element 3 and the lens element 2 is very small for the filling channel 12 for liquids. In order to allow the refractive index-adapted liquid to be filled, more space must be obtained here. To facilitate this, the filling channel 12 may be dragged down as far as possible, as is evident in this embodiment of the liquid-patient interface 1 according to the invention. So as not to collide structurally with the vacuum feedthrough 11, both channels can be offset with respect to one another, as shown in FIG. 3. Thus, they no longer lie vertically in one line (is illustrated in FIGS. 2b and 2c), but in some examples lie obliquely with respect to one another.

In a side view, FIG. 4 illustrates an ophthalmological laser therapy system 100 with a fixed liquid-patient interface 1 according to the invention, which, in turn, is docked on a patient's eye 50.

This ophthalmological laser therapy system 100 contains a main body 110, which is mounted on a movement device 180 comprising rollers. Protruding from this main body 110 there are two support structures 160, 170 that act as shafts, an articulated arm 120, 130 being disposed thereon in each case. The first articulated arm 120 contains a surgical microscope head 320 while a laser applicator 220 is disposed on the second articulated arm 130, a focused, pulsed laser radiation, which is generated in a laser source (not shown here)—in this case, a femtosecond laser source—situated in the main body 110 and which is guided via a beam guiding apparatus in support structure 170 and articulated arm 130 and a beam focusing optical unit as part of the beam guiding apparatus to the laser applicator 220, during operation. Both articulated arms 120, 130 are movable in space virtually as desired by way of their joints 140. All components of this ophthalmological examination system 100 are controlled by a control apparatus 500.

The two articulated arms 120, 130 can be interconnected by virtue of two parts of a coupling structure 150, the first part of which is disposed at the surgical microscope head 320 of the first articulated arm 120 and the second part of which is disposed at the laser applicator 220 of the second articulated arm 130, being connected to one another. So that the ophthalmological examination and therapy system 100 maintains its stability in any position of the articulated arms 120, 130 and the latter do not tilt away, for example, the articulated arms 120, 130 comprise weight balancing structures 145.

An examination beam of an apparatus for optical coherence tomography (OCT) is also guided through these articulated arms 120, 130. This is possible by application of an optical fiber in both articulated arms 120, 130. In the articulated arm 130, by application of which the pulsed laser radiation of the therapy laser apparatus is guided to the laser applicator 220, this is alternatively possible using the beam guiding apparatus with its beam focusing optical unit, which is otherwise used by the pulsed laser radiation. In this case, the examination beam is guided freely through the articulated arm 130.

Now, the liquid-patient interface 1 according to the invention is fixed by application of vacuum suction onto an applicator interface 21 of the laser applicator 220. After the fixation to the applicator interface 21, the liquid-patient interface 1 is placed with its suction lip 14 onto the patient's eye 50 and likewise fixed there by application of vacuum suction. Subsequently, it can be filled with a saline solution, such as BSS. The patient (not shown), to whom the patient's eye 50 shown here belongs, lies on a patient couch next to the ophthalmological laser therapy system 100.

In this case, the aforementioned features of the invention, which are explained in various embodiments, can be used not only in the combinations specified in an example manner but also in other combinations or on their own, without departing from the scope of the present invention.

A description of an apparatus relating to method features is analogously applicable to the corresponding method with respect to these features, while method features correspondingly represent functional features of the apparatus described.

1.-16. (canceled)

17. A liquid-patient interface that fixes the relative geometric position and orientation of a patient's eye with respect to a laser applicator of an ophthalmological laser therapy system, comprising:
a lens element and a cone element;
wherein the lens element is inserted into the cone element and permanently connected to the cone element such that the liquid-patient interface has an integral configuration;
wherein the lens element is embodied in one piece and comprises an optical zone, which has a lens function, and an envelope region, adjoining the optical zone, having a defined height not equal to zero and having an upper edge; and
wherein the upper edge of the lens element facilitates a direct connection to a laser applicator.

18. The liquid-patient interface as claimed in claim 17, wherein the upper edge of the lens element further comprises a structure that forms a mechanically stable, direct connection to an applicator interface of the laser applicator of the ophthalmological laser therapy system.

19. The liquid-patient interface as claimed in claim 18, wherein a defined structure of the upper edge of the lens element either
comprises an end face that forms a vacuum-tight connection by application of vacuum suction onto the applicator interface, wherein the vacuum suction is implemented either
by evacuating a volume that is delimited by a last optical applicator element, the applicator interface and the lens element, the applicator interface having a vacuum suction channel into the volume to this end, or
by way of the end face, the applicator interface having a vacuum suction channel that is positioned at the end face to this end, or
comprises a structure that facilitates an interlocking and/or force-fit connection to the applicator interface.

20. The liquid-patient interface as claimed in claim 18, wherein the upper edge of the lens element comprises a positive alignment structure which is configured to engage in a negative alignment structure disposed at the applicator interface.

21. The liquid-patient interface as claimed in claim 18, wherein the positive alignment structure comprises a shoulder, which is configured to engage in a negative alignment structure disposed at the applicator interface

22. The liquid-patient interface as claimed in claim 17, wherein the lens element comprises a polymer, or an optical glass.

23. The liquid-patient interface as claimed in claim 17, wherein the lens element comprises the polymer and the polymer comprises polycarbonate.

24. The liquid-patient interface as claimed in claim 17, wherein the lens element comprises the optical glass and the optical glass comprises silicon dioxide.

25. The liquid-patient interface as claimed in claim 23, wherein the lens element is made of a polymer and is formed by an injection molding method, with a gate mark being disposed outside of the optical zone.

26. The liquid-patient interface as claimed in claim 25, wherein the gate mark is disposed either
at a side of the upper edge of the lens element where the lens element has a maximum diameter, with the side being beveled such that a pin arising during molding does not exceed the maximum diameter of the upper edge, or
at the inner side of the upper edge or at the outer side of the envelope region.

27. The liquid-patient interface as claimed in claim 17, wherein a lower delimiting face of the optical zone of the lens element is embodied as an optical element of the liquid-patient interface and has a lens function.

28. The liquid-patient interface as claimed in claim 17, wherein an upper delimiting face of the optical zone of the lens element has an antireflection layer or an antireflection layer system.

29. The liquid-patient interface as claimed in claim 28, wherein the antireflection layer or the antireflection layer system is structured to suppress the reflection of radiation in at least one of the following wavelength ranges, wherein a suppression in the following wavelength range is implemented with a reflection R specified for the wavelength range:
1000 nm to 1100 nm, R<1%;
800 nm to 1200 nm, R<1%;
800 nm to 1000 nm, R<10%;
400 nm to 700 nm, with a constant reflection R over this wavelength range.

30. The liquid-patient interface as claimed in claim 17, wherein
the lens element is configured to receive, at an end side of the upper edge, illumination output coupled in from the applicator interface, or
the cone element is configured to locally form a direct contact with the applicator interface and thereby receive illumination output coupled in from the laser applicator, or both of the foregoing.

31. The liquid-patient interface as claimed in claim 17, wherein the cone element comprises a cone wall, a lower suction lip, a vacuum feedthrough, and a filling channel for liquids, wherein the vacuum feedthrough extends through the cone wall into the suction lip and the filling channel for liquids extends through the cone wall into a second volume bounded by a patient's eye, the cone wall and the lens element when vacuum suction is applied.

32. The liquid-patient interface as claimed in claim 17, wherein the lens element is adhesively bonded to the cone element with the aid of an adhesive that exerts minimal tensile or warping forces during the drying process.

33. The liquid-patient interface as claimed in claim 17, wherein the cone element comprises a collar that is structured to lengthen the cone wall.

34. The liquid-patient interface as claimed in claim 31, wherein the filling channel is disposed with such an offset in relation to the vacuum feedthrough that filling channel and vacuum feedthrough do not coincide in a view on the liquid-patient interface from above.

35. The liquid-patient interface as claimed in claim 32, wherein the filling channel is disposed with such an offset in relation to the vacuum feedthrough that filling channel and vacuum feedthrough do not coincide in a view on the liquid-patient interface from above.

36. The liquid-patient interface as claimed in claim 33, wherein the filling channel is disposed with such an offset in relation to the vacuum feedthrough that filling channel and vacuum feedthrough do not coincide in a view on the liquid-patient interface from above.

37. A production method for a liquid-patient interface for fixing the relative geometric position and orientation of a patient's eye with respect to a laser applicator of an ophthalmological laser therapy system, comprising:
manufacturing a lens element containing an optical zone, which has a lens function, and an envelope region, adjoining the optical zone, with an upper edge in one piece, and
inserting the lens element in and adhesively bonding the lens element to an integral cone element, which comprises a cone wall, a lower suction lip, a vacuum feedthrough, and a filling channel for liquids,
manufacturing the upper edge of the lens element such that upper edge facilitates a direct connection of the lens element to the laser applicator.

38. The production method as claimed in claim 37, further comprising forming a structure at the upper edge of the lens element, said structure facilitating a mechanically stable, direct connection to an applicator interface of the laser applicator.

39. The production method as claimed in claim 37, further comprising, manufacturing the lens element utilizing a polymer in injection molding method.
